(19) United States
(12) Patent Application Publication
(10) Pub. No.: US 2025/0334573 A1
(43) Pub. Date: Oct. 30, 2025

(54) LATERAL FLOW IMMUNOASSAYS COMPRISING FLUIDIC CHANNELS

(71) Applicant: ORTHO-CLINICAL DIAGNOSTICS, INC., Raritan, NJ (US)

(72) Inventor: Samantha Chang, San Marcos, CA (US)

(21) Appl. No.: 19/190,302

(22) Filed: Apr. 25, 2025

Related U.S. Application Data
(60) Provisional application No. 63/639,510, filed on Apr. 26, 2024.

(51) Int. Cl. G01N 33/543; G01N 33/531

(52) U.S. Cl. CPC G01N 33/54388; G01N 33/531

(57) ABSTRACT

The present invention relates to lateral flow immunoassay devices, including test strips, having a top layer, a bottom layer, and a middle layer, wherein the middle layer defines a fluid flow path and the top and bottom layers provide hydrophilic surfaces in contact with the middle layer. The present invention also relates to methods of detecting analytes of interest using said devices. The present invention also relates to methods of preparing immunoassay devices involving laminating a bottom layer, a middle layer, and a top layer together, wherein the middle layer defines a fluid flow path and the top and bottom layers provide hydrophilic surfaces in contact with the middle layer.

### CROSS-REFERENCE TO RELATED APPLICATIONS

This application claims the benefit of U.S. Provisional Application No. 63/639,510, filed Apr. 26, 2024, which is incorporated by reference herein in its entirety.

### TECHNICAL FIELD

The present invention relates to lateral flow immunoassay devices, including test strips, having a top layer, a bottom layer, and a middle layer, wherein the middle layer defines a fluid flow path and the top and bottom layers provide hydrophilic surfaces in contact with the middle layer. The present invention also relates to methods of detecting analytes of interest using said devices. The present invention also relates to methods of preparing immunoassay devices involving laminating a bottom layer, a middle layer, and a top layer together, wherein the middle layer defines a fluid flow path and the top and bottom layers provide hydrophilic surfaces in contact with the middle layer.

### BACKGROUND

Traditional lateral flow strips for use in, e.g., diagnostic immunoassays have limitations which have not been addressed in the art.

For example, in traditional immunoassay flow strips, a fluid sample is applied to sample pad from which the fluid sample may flow toward various reagents and the like.

However, some volume of the fluid sample is necessarily retained in the sample pad.

There is an unmet need for more efficient lateral flow devices which do not waste fluid sample and which can be reused by washing the device between uses.

The present invention avoids the need for sample pads altogether while reducing sample waste and allowing for wash cycles between assays.

### BRIEF SUMMARY

The following aspects and embodiments thereof described and illustrated below are meant to be exemplary and illustrative, not limiting in scope.

In an aspect, an immunoassay device is provided. The immunoassay device comprises a bottom layer, a middle layer, and a top layer, wherein the middle layer is provided between the top layer and the bottom layer and the top, middle, and bottom layers are laminated together,
wherein the top layer comprises an inlet defining a sample application zone, wherein said sample application zone is in fluid communication with the middle and bottom layers,
wherein a surface of the top layer in contact with the middle layer comprises a hydrophilic composition,
wherein a surface of the bottom layer in contact with the middle layer comprises a hydrophilic composition,
wherein the middle layer comprises a first adhesive surface in contact with the top layer and a second adhesive surface in contact with the bottom layer,
wherein the middle layer defines a flow pathway extending from the sample application zone and the thickness of the middle layer defines a height of the flow pathway, and
wherein at least one capture reagent is provided in the flow pathway.

In certain embodiments, the capture reagent is a polypeptide.

In certain embodiments, the capture reagent is a protein.

In certain embodiments, the capture reagent is an antibody.

In certain embodiments, the capture reagent is an antibody selected from the group consisting of Flu A antibody, Flu B antibody, RSV antibody, and SARS antibody.

In certain embodiments, the capture reagent is a hapten-carrier protein selected from the group consisting of fentanyl-BSA, oxycodone-BSA, and buprenorphine-BSA.

In certain embodiments, at least one reporter conjugate is provided in the flow pathway between the at least one capture reagent and the sample application zone.

In certain embodiments, the at least one reporter conjugate is selected from the group consisting of Flu A antibody conjugated to detectable particles, Flu B antibody conjugated to detectable particles, RSV antibody conjugated to detectable particles, SARS antibody conjugated to detectable particles, fentanyl antibody conjugated to detectable particles, oxycodone antibody, and buprenorphine antibody conjugated to detectable particles. In certain embodiments, the detectable particles are Europium particles.

In certain embodiments, the middle layer comprises polyester and an acrylic adhesive.

In an aspect, method of preparing an immunoassay device is provided. The method of preparing an immunoassay device comprises providing a top layer comprising a first surface comprising a hydrophilic composition, wherein the top layer comprises an opening,
providing a bottom layer comprising a first surface comprising a hydrophilic composition,
providing a middle layer between the top layer and the bottom layer such that the middle layer is in contact with the first surface of the top layer and the first surface of the bottom layer,
wherein the middle layer comprises two surfaces, each comprising an adhesive composition,
prior to providing the middle layer between the top and bottom layers, cutting the middle layer to form a channel through the middle layer, and
after providing the middle layer between the top and bottom layers, applying compressive force such that the top, middle, and bottom layers are laminated together, thereby providing the opening in the top layer in fluid communication with the channel.

In certain embodiments, the hydrophilic composition of the first surface of the bottom layer is nitrocellulose.

In certain embodiments, the bottom layer is nitrocellulose.

In certain embodiments, the method further comprises depositing a capture reagent on the nitrocellulose.

In certain embodiments, the capture reagent is a polypeptide.

In certain embodiments, the capture reagent is a protein.

In certain embodiments, the capture reagent is an antibody.

In certain embodiments, the capture reagent is an antibody selected from the group consisting of Flu A antibody, Flu B antibody, RSV antibody, and SARS antibody.

In certain embodiments, a conjugate is deposited in the flow pathway between the capture reagent and the sample application zone.

In certain embodiments, the reporter conjugate is selected from the group consisting of Flu A antibody conjugated to detectable particles, Flu B antibody conjugated to detectable particles, RSV antibody conjugated to detectable particles, SARS antibody conjugated to detectable particles, fentanyl antibody conjugated to detectable particles, oxycodone antibody, and buprenorphine antibody conjugated to detectable particles. In certain embodiments, the detectable particles are Europium particles.

In certain embodiments, the middle layer comprises polyester and an acrylic adhesive.

In an aspect, method of detecting presence of an analyte of interest in a fluid sample is provided. The method of detecting presence of an analyte of interest in a fluid sample comprises
applying the fluid sample to a sample application zone provided on an immunoassay device,
wherein the immunoassay device comprises a laminated structure comprising a bottom layer, a middle layer, and a top layer, wherein the middle layer is provided between the top layer,
wherein the top layer comprises an opening to the sample application zone, wherein said sample application zone is in fluid communication with the middle and bottom layers,
wherein a surface of the top layer in contact with the middle layer comprises a hydrophilic composition,
wherein a surface of the bottom layer in contact with the middle layer comprises a hydrophilic composition,
wherein the middle layer comprises a first adhesive surface in contact with the top layer and a second adhesive surface in contact with the bottom layer,
wherein the middle layer defines a flow pathway extending from the sample application zone and the thickness of the middle layer defines a height of the flow pathway, and
wherein at least one capture reagent capable of binding the analyte of interest is provided in the flow pathway,
wherein the applied fluid sample flows from the sample application zone to a terminus of the flow pathway.

In certain embodiments, the at least one capture reagent is a protein.

In certain embodiments, the at least one capture reagent is an antibody.

In certain embodiments, the at least one capture reagent is an antibody selected from the group consisting of Flu A antibody, Flu B antibody, RSV antibody, and SARS antibody, fentanyl antibody, oxycodone antibody, and buprenorphine antibody.

In certain embodiments, at least one conjugate is provided in the flow pathway between the capture reagent and the sample application zone.

In certain embodiments, the at least one reporter conjugate is selected from the group consisting of Flu A antibody conjugated to detectable particles, Flu B antibody conjugated to detectable particles, RSV antibody conjugated to detectable particles, SARS antibody conjugated to detectable particles, fentanyl antibody conjugated to detectable particles, oxycodone antibody, and buprenorphine antibody conjugated to detectable particles. In certain embodiments, the detectable particles are Europium particles.

In certain embodiments, the middle layer comprises polyester and an acrylic adhesive.

### BRIEF DESCRIPTION OF THE DRAWINGS

FIG. 1 illustrates an exemplary, non-limiting assembly of a device of the present invention.

FIG. 2A illustrates an exemplary, non-limiting embodiment of a device of the present invention wherein spotted capture reagent is provided in the fluidic path. A sample port 21 directs sample via the highlighted path 22 toward the fluidic channel 23 formed in the structured nitrocellulose bottom layer. Spotted capture reagents 24 are provided in the fluidic channel 23. An absorbent pad 25 is provided downstream of the fluidic channel 23. Additionally, the embodiment of FIG. 2A includes an ablated portion of nitrocellulose 26.

FIG. 2B illustrates an exemplary, non-limiting embodiment of a device of the present invention wherein spotted capture reagent is provided in the fluidic path and reporter/detection conjugate is provided upstream of the spotted capture reagent within the fluidic path. The device of FIG. 2B is similar to that of FIG. 2A, but the spotted reporter/detection conjugate 27 is provided upstream of the capture reagent 24 such that analyte in an applied sample binds to the reporter/detection conjugate 27 first.

FIG. 3A illustrates a preparation of multiple device assemblies according to the present invention, wherein a single lamination step can be performed prior to cutting the assembly into individual strips, thereby streamlining manufacturing.

FIG. 3B shows a device of the present invention and an immunoassay cassette in which the device of the present invention may be provided for use with compatible immunoassay platforms or instruments.

FIG. 3C illustrates the flowpath of a fluid sample ten minutes after being administered to the sample application zone of a device of the present invention.

FIGS. 4A-4F illustrate exemplary, non-limiting embodiments of the device of the present invention having various fluid pathway configurations.

FIG. 5 illustrates an exemplary, non-limiting assembly of a device of the present invention.

FIG. 6 is a side-by-side comparison of a hydrophilic channel slide assembly of the present invention against a glass fiber assembly.

FIG. 7 shows a comparison of signal responses obtained using strips having a hydrophilic channels (e.g., hydrophilic sample ports) versus signal responses obtained using strips having glass fiber application zones.

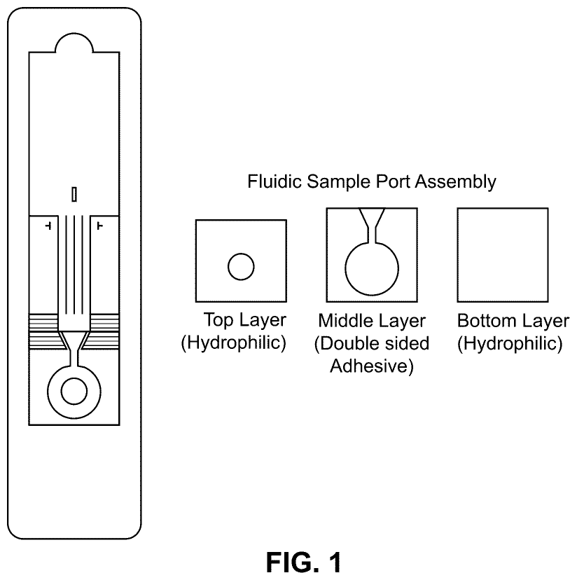

### DETAILED DESCRIPTION

Where a range of values is provided, it is intended that each intervening value between the upper and lower limit of that range and any other stated or intervening value in that stated range is encompassed within the disclosure. For example, if a range of 1 μm to 8 μm is stated, it is intended that 2 μm, 3 μm, 4 μm, 5 μm, 6 μm, and 7 μm are also explicitly disclosed, as well as the range of values greater than or equal to 1 μm and the range of values less than or equal to 8 μm.

The singular forms “a,” “an,” and “the” include plural referents unless the context clearly dictates otherwise. Thus, for example, reference to a “polymer” includes a single polymer as well as two or more of the same or different polymers, reference to an “excipient” includes a single excipient as well as two or more of the same or different excipients, and the like.

The disjunctive “or” is inclusive, unless otherwise specified. For example, “X or Y” means “X, Y, or both X and Y” unless otherwise specified.

The word “about” when immediately preceding a numerical value means a range of plus or minus 10% of that value, e.g., “about 50” means 45 to 55, “about 25,000” means 22,500 to 27,500, etc., unless the context of the disclosure indicates otherwise, or is inconsistent with such an interpretation. For example in a list of numerical values such as “about 49, about 50, about 55, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein.

As used herein with respect to a substrate or support, the terms “immobilizing” or “immobilized” include covalent conjugation, non-specific association, ionic interactions and other means of adhering a substance (e.g., a polymer, a copolymer, a binding moiety) to a substrate or support, i.e., a surface of a substrate or support.

As used herein, the term “antibody” means a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, for example, as intact immunoglobulins or as a number of well characterized fragments originally produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. The “Fc” portion of an antibody refers to that portion of an immunoglobulin heavy chain that comprises one or more heavy chain constant region domains, CH₁, CH₂ and CH₃, but does not include the heavy chain variable region.

The devices, kits and methods of the present disclosure can comprise, consist essentially of, or consist of, the components or steps disclosed.

All ranges disclosed herein include all subranges contained therein, as well as all discreet values contained therein. Additionally, all ranges disclosed herein are inclusive of their endpoints, unless otherwise specified. For example, “X to Y” means “greater than or equal to X and less than or equal to Y” unless otherwise specified.

When used to describe the amounts of components of a composition, all percentages, parts and ratios are based upon the total weight of the composition, unless otherwise specified.

All measurements made are at about 25° C., unless otherwise specified. Additionally, all measurements made are at about 1 atm of pressure, unless otherwise specified.

By reserving the right to proviso out or exclude any individual members of any such group, including any sub-ranges or combinations of sub-ranges within the group, that can be claimed according to a range or in any similar manner, less than the full measure of this disclosure can be claimed for any reason. Further, by reserving the right to proviso out or exclude any individual substituents, analogs, compounds, ligands, structures, or groups thereof, or any members of a claimed group, less than the full measure of this disclosure can be claimed for any reason.

Throughout this disclosure, various patents, patent applications and publications are referenced. The disclosures of these patents, patent applications and publications in their entireties are incorporated into this disclosure by reference in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

As used herein to indicate a location of a system component, the direction of fluid flow, or otherwise refer to a point or relative position within a device or system described herein, the term “downstream” means either at a position further away from an application zone (e.g., a sample application zone) or closer to a detection zone (e.g., an analyte detection zone) or region of interest than a reference point or system component, or in a direction away from a sample application zone or toward a region of interest. Conversely, “upstream” means either at a position closer to a sample application zone or further from a region of interest than a reference point of system component, or in a direction toward a sample application zone or away from a region of interest. By way of illustration and without limitation, a component B is downstream of a component A if component B is between component A and a region of interest. Also by way of illustration and without limitation, a component C is upstream of component A if it is between component A and a sample application zone.

In an aspect of the lateral flow device of the present invention, a top layer and a bottom layer are provided with a middle layer provided between the top layer and the bottom layer. In an aspect, the top layer, middle layer, and bottom layer are laminated together to form a lateral flow device (e.g., a test strip) of the present invention.

In an aspect, a surface of the top hydrophilic film layer forms the top side of the fluidic channel or path.

In certain embodiments, the top layer comprises an opening or inlet. The opening or inlet provided in the top hydrophilic film layer provides access to the middle layer and bottom layer. According to a method of the present invention, a sample can be introduced via the opening or inlet.

In an aspect of the present invention, the provision of hydrophilic surfaces on either side of a middle fluidic channel or pathway causes flow of aqueous sample.

In certain embodiments, the middle layer comprises two surfaces on opposite sides, each surface comprising an adhesive.

In certain embodiments, the middle layer comprises two surfaces on opposite sides, each surface comprising an adhesive which is non-migratory and inert. Preferably, the adhesive is easily die-cut.

In certain embodiments, the middle layer consists of an inert film wherein the middle layer is coated on opposite sides with an adhesive. In certain embodiments, the inert film is easily die-cut.

In certain embodiments, the middle layer is a polyester layer having two surfaces on opposite sides, wherein said opposite sides are coated with an adhesive.

In certain embodiments, the middle layer is a polyester layer which is coated with an acrylic adhesive on opposite sides.

In certain embodiments, the middle layer consists essentially of an adhesive.

In certain embodiments, the middle layer consists of an adhesive.

In certain embodiments, the middle layer is an acrylic adhesive.

In an aspect, the thickness of the middle layer defines the height of the fluidic channel(s) or path(s). In certain embodiments, the middle layer has a thickness of about 0.001 mm to about 1.0 mm, or about 0.0025 mm to about 0.50 mm, or about 0.005 mm to about 0.25 mm, or about 0.01 mm to about 0.10 mm.

In certain embodiments, the top layer is a hydrophilic film having a thickness of about 0.001 mm to about 0.1 mm, or about 0.0005 mm to about 0.075 mm, or about 0.001 mm to about 0.05 mm, or about 0.0025 mm to about 0.025 mm.

In certain embodiments, the top layer comprises a hydrophilic film.

In certain embodiments, the top layer comprises a hydrophilic film on a surface of the top layer in contact with the middle layer.

In certain embodiments, the top layer is a hydrophilic film.

In certain embodiments, the top layer is a hydrophilic film free of surfactants.

In certain embodiments, the bottom layer comprises nitrocellulose.

In certain embodiments, the bottom layer consists essentially of nitrocellulose.

In certain embodiments, the bottom layer consists of nitrocellulose.

In certain embodiments, one or more channels are formed in the bottom layer. For example, the one or more channels formed in the bottom layer may be formed by etching or laser ablation.

In certain embodiments wherein one or more channels are formed in the bottom layer, said one or more channels in the bottom layer may align with one or more channels formed in the middle layer.

In certain embodiments, the fluidic path is formed via removal of material from the middle layer.

In certain embodiments, the fluidic path is formed by ablating the middle layer prior to lamination.

In certain embodiments, the fluidic path is formed by lasering the middle layer prior to lamination.

In certain embodiments, the fluidic path is formed by die-cutting the middle layer prior to lamination.

In certain embodiments, the fluidic path comprises a single lane.

Alternatively, the fluidic path may comprise a plurality of lanes. In certain embodiments, the fluidic path comprises 2, 3, 4, 5, 6, 7, 8, 9, 10, or more lanes.

In certain embodiments, the fluidic path comprises a plurality of parallel lanes. In certain embodiments, the fluidic path comprises 2, 3, 4, 5, 6, 7, 8, 9, 10, or more parallel lanes.

In certain embodiments, the fluidic path comprises a single, nonlinear fluidic path.

In certain embodiments, the fluidic path comprises two or more nonlinear fluidic paths.

For example, the device of the present invention may comprise one or more nonlinear fluidic paths having one or more turns, angles, or curves. In an aspect, the geometry of the fluidic path may be designed to modify or control the rate of sample flow downstream from the sample application zone.

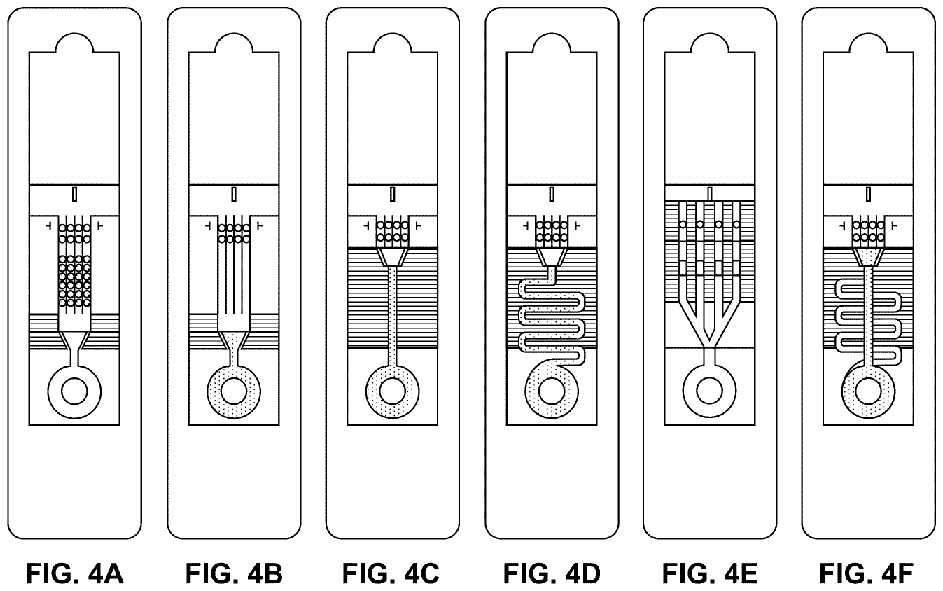

FIGS. 4A-4F show exemplary, non-limiting embodiments of the present invention having different configurations of fluid channels and pathways.

FIGS. 4A-4F show ablation of nitrocellulose from the bottom (support) layer.

In the exemplary, non-limiting embodiment of FIG. 4B, the area surrounding the sample application zone is covered by the hydrophilic surface of the top layer, whereas the remainder of the fluidic paths are not covered. The hydrophilic surface of the top layer over the sample application zone causes flow of the sample toward the fluidic paths.

FIG. 4C shows an exemplary, non-limiting embodiment having hydrophilic paths extending to deposited reagents/conjugates.

FIG. 4D similarly shows an exemplary, non-limiting embodiment wherein a hydrophilic path extending to deposited reagents/conjugates. The path of FIG. 4D has a plurality of turns provided, causing applied sample to take longer to reach the deposited reagents/conjugates.

FIG. 4E shows an exemplary, non-limiting embodiment in which the nitrocellulose is ablated proximal to the deposited reagents/conjugates.

FIG. 4F shows an exemplary, non-limiting embodiment in which two different fluid pathways are provided in a stack to allow for controlled and sequential flow of sample and reagents/conjugates onto the bottom layer (e.g., a nitrocellulose support). In such embodiments having multiple fluid pathways having different lengths, the shorter path would allow sample antigen to flow and bind to a capture antibody first, then the conjugate (e.g., reporter molecule) dispensed or deposited within the longer fluidic path would follow to form a sandwich signal on the capture spots. In certain other embodiments, wash reagent may be dispensed within the fluidic channels or paths to wash the channels or paths once the sample and conjugate has flowed through the shorter fluidic path.

In an aspect, a surface of the bottom hydrophilic film layer forms the bottom side of the fluidic channel.

In an aspect of the present invention, the top layer and the middle layer have length and width dimensions narrower than the bottom layer. Such a configuration prevents overflow of fluid sample outside the perimeter of the intended fluidic path(s).

In certain embodiments, the opening or inlet may be provided in fluid communication with an automated fluidic system or manually operated fluidic system to introduce sample into the device. In such embodiments, the fluidic system may comprise one or more pumps, one or more reservoirs, and/or one or more valves.

In certain embodiments, one or more reagents may be provided in the fluidic path(s).

In certain embodiments, the same reagent is provided in all fluidic paths.

In certain embodiments, different reagents are provided in different fluidic paths for multiplex analysis of a provided sample.

In certain embodiments, the reagents are capture reagents capable of binding to analytes of interest suspected of being present in a provided sample.

In certain embodiments, one or more reporter or detection conjugates may be provided in the fluidic path(s).

In certain embodiments, the same reporter or detection conjugate is provided in all fluidic paths.

In certain embodiments, different reporter or detection conjugates are provided in different fluidic paths for multiplex analysis of a provided sample.

In certain embodiments comprising reporter or detection conjugates provided in the fluidic path(s), said reporter or detection conjugates are provided upstream of any provided capture reagents (i.e., between the sample application zone and the provided capture reagent(s)).

Reagents (e.g., capture reagents) may be provided by spotting said reagents within the fluidic path(s). A reporter or detection conjugate may similarly be spotted in the fluidic path(s).

In certain embodiments, reagents and/or conjugates may be bound to a bottom layer (e.g., a nitrocellulose layer). In such embodiments, the reagents or conjugates may be dispensed in nanoliter volumes.

Where a reagent such as a capture reagent is provided in the fluidic path(s), sample administered to the sample application zone flows downstream from the sample application zone and toward a support (e.g., a nitrocellulose support) via the fluidic path(s). Sample thus mixes with the capture reagent within the fluidic path(s) before flowing onto the support.

Similarly, where one or more reporter or detection conjugates are provided in the fluidic path(s), sample flows through the fluidic path(s), thereby mixing first with the reporter or detection conjugate(s) and subsequently with provided reagents (e.g., capture reagents) before flowing onto the support.

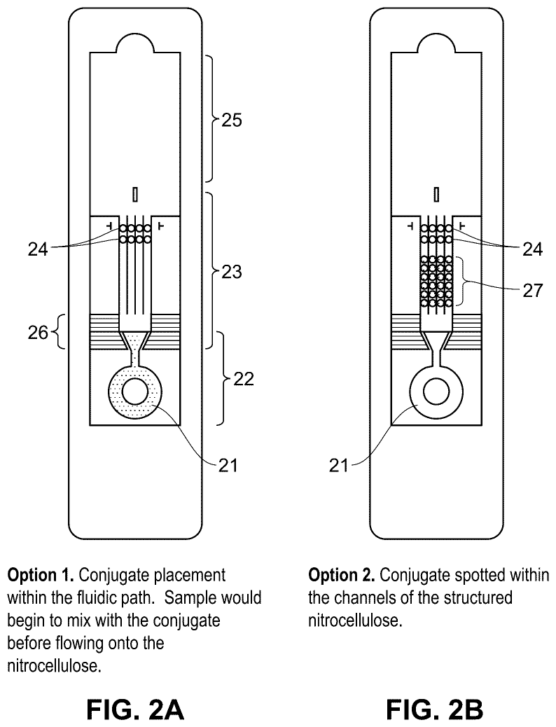

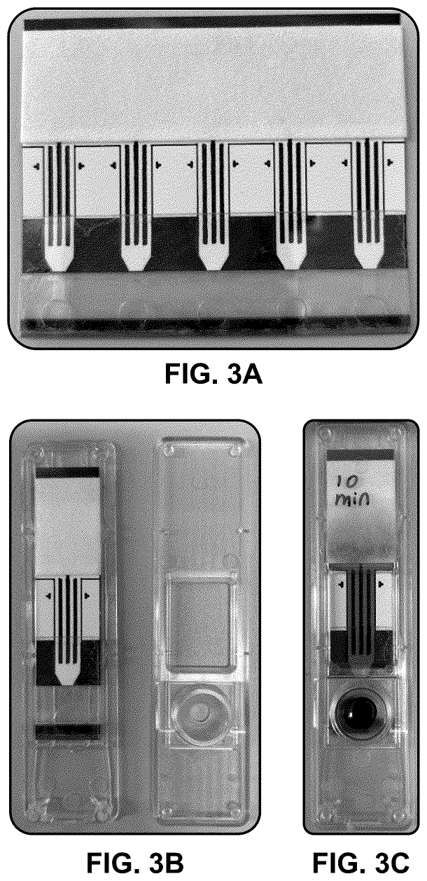

In certain embodiments, an absorbent pad may be provided downstream of the fluidic channel(s) or path(s) to collect excess sample. See, e.g., FIG. 2A and FIGS. 3A-3C.

In certain embodiments, multiple assemblies can be stacked in a single test strip to provide multiple fluidic paths. For instance, in certain embodiments, a hydrophilic film similar to or identical to the top layer may be provided between two middle layers, where each middle layer defines a different fluid pathway. Different capture reagents and/or reporter/detection conjugates may be provided in each fluid channel defined by the different middle layers.

Further aspects of the present subject matter will be apparent to persons of ordinary skill in the art based on the following non-limiting Examples.

### Example 1

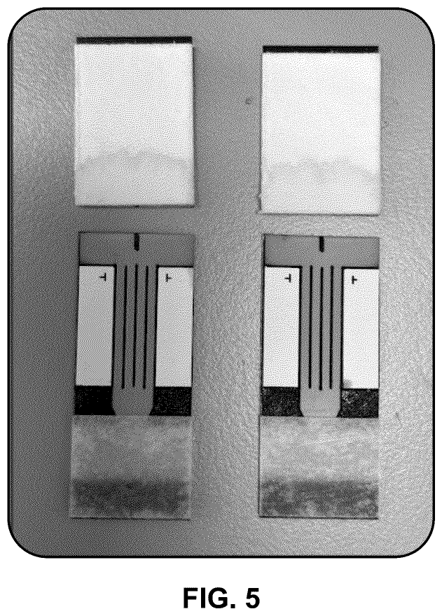

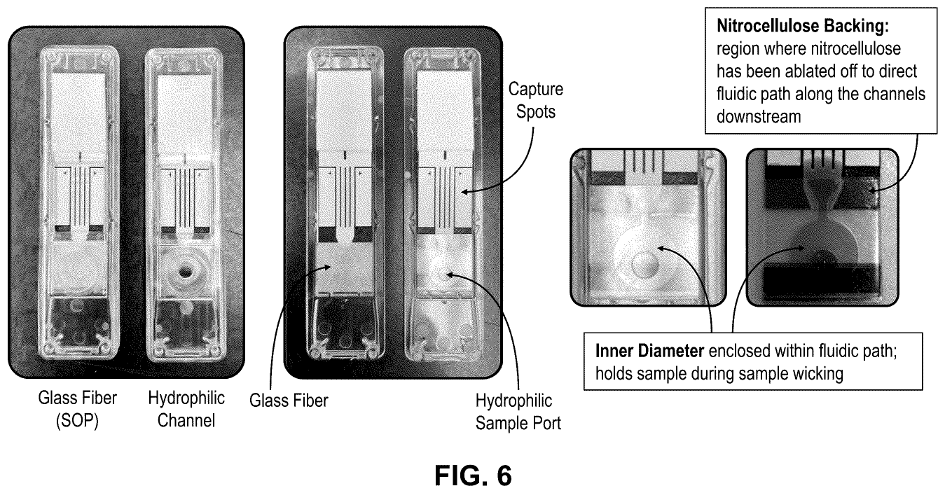

Strips were assembled and cut at the interface between the absorbent pad and the nitrocellulose. FIG. 5 shows the prepared assembly.

Absorbents pads were weighed to determine the baseline weight of the absorbent pad in the absence of sample.

120 uL of sample was added to each strip design and allowed to flow for 15 minutes. After 15 minutes elapsed, each strip was cut at the absorbent pad/nitrocellulose interface and weighed.

The difference between the wet and dry weight of the absorbent pad represents the sample volume migration. Results are shown in Table 1.

TABLE 1

| | Dry | | Hydrophilic |
|---|---|---|---|
| | Weight | Glass | Sample |
| Replicate | (g) | Fiber | Port |
| 1 | 0.1715 | 0.2371 | 0.2453 |
| 2 | 0.1692 | 0.2385 | 0.2472 |
| 3 | 0.1723 | 0.2332 | 0.2477 |
| 4 | 0.1722 | 0.2346 | 0.2437 |
| 5 | 0.1673 | | |
| 6 | 0.1621 | | |
| 7 | 0.1699 | | |
| 8 | 0.1675 | | |
| 9 | 0.1708 | | |
| 10 | 0.1703 | | |
| Mean | 0.1693 | 0.2359 | 0.2460 |
| SD | 0.0031 | 0.0024 | 0.0018 |
| CV | 1.81% | 1.01% | 0.75% |
| Sample Vol (uL) | | 66.5 | 76.7 |

As shown in Table 1, the hybrid design with the hydrophilic sample port delivered 10 uL more sample volume than a traditional lateral strip.

### Example 2

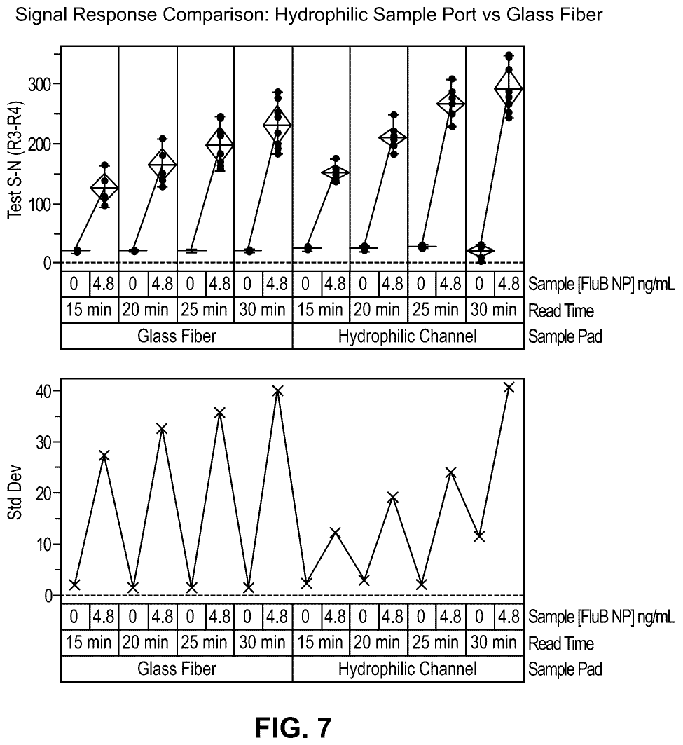

FIG. 7 shows a comparison of signal responses obtained using strips having a hydrophilic channels (e.g., hydrophilic sample ports) versus signal responses obtained using strips having glass fiber application zones.

As the assay progressed, more sample was released in the hydrophilic design and signal differences between the two designs increased.

Positive and negative samples were tested in both strip designs to compare signal output.

Table 2 shows the signals obtained after 15, 20, 25, and 30 minutes following application of negative sample (0 ng/ml) and positive sample (4.8 ng/ml) using both glass fiber and hydrophilic channel assemblies.

TABLE 2

| | Sample | | Mean | | SD | | |
|---|---|---|---|---|---|---|---|
| Read | [FluB NP] | | | Hydrophilic | | Hydrophilic | Signal |
| Time | ng/mL | n | Glass Fiber | Channel | Glass Fiber | Channel | Delta |
| 15 | 0 | 8 | 20.6 | 24.7 | 2.0 | 2.3 | 4.1 |
| 20 | 0 | 8 | 20.6 | 25.1 | 1.3 | 2.9 | 4.5 |
| 25 | 0 | 8 | 20.3 | 27.1 | 1.4 | 2.1 | 6.7 |
| 30 | 0 | 8 | 21.0 | 21.0 | 1.6 | 11.4 | 0.0 |
| 15 | 4.8 | 8 | 126.1 | 150.9 | 27.5 | 12.3 | 24.7 |
| 20 | 4.8 | 8 | 164.7 | 210.0 | 32.5 | 19.3 | 45.2 |
| 25 | 4.8 | 8 | 197.7 | 266.3 | 35.8 | 24.0 | 68.7 |
| 30 | 4.8 | 8 | 230.5 | 291.8 | 40.1 | 40.6 | 61.3 |

What is claimed:

1. An immunoassay device, comprising a bottom layer, a middle layer, and a top layer,
wherein the middle layer is provided between the top layer and the bottom layer and the top, middle, and bottom layers are laminated together,
wherein the top layer comprises an inlet defining a sample application zone, wherein said sample application zone is in fluid communication with the middle and bottom layers,
wherein a surface of the top layer in contact with the middle layer comprises a hydrophilic composition,
wherein a surface of the bottom layer in contact with the middle layer comprises a hydrophilic composition,
wherein the middle layer comprises a first adhesive surface in contact with the top layer and a second adhesive surface in contact with the bottom layer,
wherein the middle layer defines a flow pathway extending from the sample application zone and the thickness of the middle layer defines a height of the flow pathway, and
wherein at least one capture reagent is provided in the flow pathway.

2. The immunoassay device of claim 1, wherein the capture reagent is a polypeptide.

3. The immunoassay device of claim 1, wherein the capture reagent is a protein.

4. The immunoassay device of claim 1, wherein the capture reagent is an antibody.

5. The immunoassay device of claim 1, wherein the capture reagent is an antibody selected from the group consisting of Flu A antibody, Flu B antibody, RSV antibody, and SARS antibody, fentanyl antibody, oxycodone antibody, and buprenorphine antibody.

6. The immunoassay device of claim 1, wherein at least one reporter conjugate is provided in the flow pathway between the at least one capture reagent and the sample application zone.

7. The immunoassay device of claim 6, wherein the at least one reporter conjugate is an antibody conjugated to Europium particles, wherein the antibody is selected from the group consisting of Flu A antibody, Flu B antibody, RSV antibody, SARS antibody, fentanyl antibody, oxycodone antibody, and buprenorphine antibody.

8. The immunoassay device of claim 1, wherein the middle layer comprises polyester and an acrylic adhesive.

9. A method of preparing an immunoassay device, comprising:
providing a top layer comprising a first surface comprising a hydrophilic composition, wherein the top layer comprises an opening,
providing a bottom layer comprising a first surface comprising a hydrophilic composition,
providing a middle layer between the top layer and the bottom layer such that the middle layer is in contact with the first surface of the top layer and the first surface of the bottom layer,
wherein the middle layer comprises two surfaces, each comprising an adhesive composition,
prior to providing the middle layer between the top and bottom layers, cutting the middle layer to form a channel through the middle layer, and
after providing the middle layer between the top and bottom layers, applying compressive force such that the top, middle, and bottom layers are laminated together, thereby providing the opening in the top layer in fluid communication with the channel.

10. The method of claim 9, wherein the hydrophilic composition of the first surface of the bottom layer is nitrocellulose.

11. The method of claim 9, wherein the bottom layer is nitrocellulose.

12. The method of claim 10, further comprising depositing a capture reagent on the nitrocellulose.

13. The method of claim 12, wherein the capture reagent is a polypeptide.

14. The method of claim 12, wherein the capture reagent is a protein.

15. The method of claim 12, wherein the capture reagent is an antibody.

16. The method of claim 9, wherein the capture reagent is an antibody selected from the group consisting of Flu A antibody, Flu B antibody, RSV antibody, and SARS antibody, fentanyl antibody, oxycodone antibody, and buprenorphine antibody.

17. The method of claim 12, wherein a conjugate is deposited in the flow pathway between the capture reagent and the sample application zone.

18. The method of claim 17, wherein the reporter conjugate is an antibody conjugated to Europium particles, wherein the antibody is selected from the group consisting of Flu A antibody, Flu B antibody, RSV antibody, SARS antibody, fentanyl antibody, oxycodone antibody, and buprenorphine antibody.

19. The method of claim 9, wherein the middle layer comprises polyester and an acrylic adhesive.

20. A method of detecting presence of an analyte of interest in a fluid sample, comprising:
applying the fluid sample to a sample application zone provided on an immunoassay device,
wherein the immunoassay device comprises a laminated structure comprising a bottom layer, a middle layer, and a top layer, wherein the middle layer is provided between the top layer,
wherein the top layer comprises an opening to the sample application zone, wherein said sample application zone is in fluid communication with the middle and bottom layers,
wherein a surface of the top layer in contact with the middle layer comprises a hydrophilic composition,
wherein a surface of the bottom layer in contact with the middle layer comprises a hydrophilic composition,
wherein the middle layer comprises a first adhesive surface in contact with the top layer and a second adhesive surface in contact with the bottom layer,
wherein the middle layer defines a flow pathway extending from the sample application zone and the thickness of the middle layer defines a height of the flow pathway, and
wherein at least one capture reagent capable of binding the analyte of interest is provided in the flow pathway,
wherein the applied fluid sample flows from the sample application zone to a terminus of the flow pathway.

21. The method of claim 20, wherein the at least one capture reagent is a protein.

22. The method of claim 20, wherein the at least one capture reagent is an antibody.

23. The method of claim 22, wherein the at least one capture reagent is an antibody selected from the group consisting of Flu A antibody, Flu B antibody, RSV antibody, and SARS antibody, fentanyl antibody, oxycodone antibody, and buprenorphine antibody.

24. The method of claim 20, wherein at least one conjugate is provided in the flow pathway between the capture reagent and the sample application zone.

25. The method of claim 20, wherein the at least one reporter conjugate is an antibody conjugated to Europium particles, wherein the antibody is selected from the group consisting of Flu A antibody, Flu B antibody, RSV antibody, SARS antibody, fentanyl antibody, oxycodone antibody, and buprenorphine antibody.

26. The method of claim 20, wherein the middle layer comprises polyester and an acrylic adhesive.
